(19) United States
(12) Patent Application Publication
(10) Pub. No.: US 2019/0191995 A1
(43) Pub. Date: Jun. 27, 2019

(54) Systems and Methods for Capturing and Analyzing Pupil Images to Determine Toxicology and Neurophysiology

(71) Applicant: PupilScan Corporation, Princeton, NJ (US)

(72) Inventors: Vincent J. Giovinazzo, Staten Island, NY (US); Devin F. Hosea, Princeton, NJ (US); William F. Vaughn, Sebastopol, CA (US)

(21) Appl. No.: 16/045,436

(22) Filed: Jul. 25, 2018

Related U.S. Application Data
(60) Provisional application No. 62/538,306, filed on Jul. 28, 2017.

(51) Int. Cl. A61B 3/14; A61B 3/11; A61B 3/00; A61B 5/00

(52) U.S. Cl. CPC A61B 3/145; A61B 3/112; A61B 3/0025; A61B 5/4845; A61B 2560/0431; A61B 5/7264; A61B 5/4836; A61B 3/0041; A61B 5/4064

(57) ABSTRACT

Disclosed are systems and methods for capturing a pupillary light reflex (PLR) by capturing images of a subject's pupil, for example using a smartphone, extracting image data to determine PLR and classifying the PLR to provide an analytical output, such as a diagnosis or prognosis, of a neurological or psychiatric brain condition.

### CROSS-REFERENCE TO RELATED PATENT APPLICATION

This patent application claims a benefit to the filing date of U.S. Provisional Patent Application Ser. No. 62/538,306, titled “Methods for Capturing and Analyzing Pupil Images to Determine Toxicology and Neurophysiology,” that was filed on Jul. 28, 2017. The disclosure of U.S. 62/538,306 is incorporated by reference herein in its entirety.

### FIELD OF THE DISCLOSURE

Disclosed are systems, methods and apparatus for capturing pupillary light reflex (PLR) and using the PLR for analytical methods including diagnosis of a level of chemical substances in a subject and treatment of disease.

### BACKGROUND OF THE DISCLOSURE

Observations and measurements of pupil size date to Archimedes (287-212 B.C.) and Galileo (1564-1642). The simple reflex arc of pupillary contractions to light has been studied by physicians and scientists for centuries. Retinal ganglion cells project afferent fibers via the optic nerve, optic chiasm and optic tracts to synapse with intercalated neurons in the midbrain pretectum. These pretectal neurons conduct the afferent information to the nuclei that act on the sphincter muscle of the iris in the oculomotor complex. Preganglionic efferent fibers from this nucleus travel with the third cranial nerve to the ciliary ganglion and, finally, postganglionic short ciliary fibers reach the muscle cells of the iris sphincter causing constriction of the pupil. The process of pupillary contractions to light can be easily summarized, but the vast interconnections hidden within the brain exert subtle but identifiable influences on the pupillary light reflex (PLR).

Pupilometers capable of capturing PLR are known in the art. Exemplary United

States patents are:

U.S. Pat. No. 6,116,736—Pupilometer with Pupil Irregularity Detection Capability;

U.S. Pat. No. 6,820,979—Pupilometer with Pupil Irregularity Detection, Pupil Tracking, and Pupil Response Detection Capability, Glaucoma Screening Capability, Intracranial Pressure Detection Capability, and Ocular Aberration Measurement Capability;

U.S. Pat. No. 7,967,442—Methods, Systems, and Devices for Monitoring Anisocoria and Asymmetry of Pupillary Reaction to Stimulus; and

U.S. Pat. No. 8,393,734—Pupillary Screening Method and System. Conventional pupilometers are designated devices fit for capturing PLR and are usually held in contact with the eye to capture an image of the pupil. Furthermore, commercial or consumer versions of pupilometers typically do not provide any interpretation of PLR. Each of U.S. Pat. Nos. 6,116,736; 6,820,979; 7,967,442 and 8,393,734 is incorporated by reference herein in its entirety.

One current method for detecting chemical substances in a test subject is a fluid-based test. Commercially available assay tests use antibodies to detect the presence of certain substances in the bloodstream. Three disadvantages of assay tests:

1) the tests have difficulty detecting purely synthetic toxins such as fentanyl or methadone;

2) the tests reveal drug content in the blood outside of the blood-brain barrier, not neurological state or blood content inside the blood-brain barrier; and 3) the tests take time and are invasive. Fluid-based tests require fluid acquisition and then testing. Often in excess of 15 minutes is required to run the test and obtain results. By contrast, the methods that are disclosed herein below provide an immediate reading of actual neurological intoxication and provide a more relevant result, present neurological state vs. blood, saliva or urine toxin levels, in a non-invasive procedure. The methods disclosed herein below are easily used on unconscious patients in almost any environment, such as outside of a clinical setting.

A second current method for detecting chemical substances in a test subject is a mass spectrometry test, currently viewed as the “gold standard” for detection and identification of chemical substances. Fluid samples usually must be sent to a laboratory for mass spectrometry analysis. The wait time for results is typically at least 12 hours and may be more than one week when fluid samples are sent in batches. In addition to a slow response time, mass spectrometry tests have a drawback akin to the assay tests. The tests measure toxin levels in bodily fluids, not toxin levels past the blood-brain barrier or, more importantly, the neurological state that those toxin levels induce. In many cases, these are not identical. Rather, the neurological state of the patient is inferred from the toxin level in the bodily fluid.

### BRIEF SUMMARY

An object of the disclosure below is to provide methods, systems and apparatuses effective to:
Determine methods for triggering PLR in order to observe pupil behavior. The methods include, but are not limited to, various illumination sequences.
Obtain sufficiently clear images to allow accurate pupil measurement at high frequency.
Identifying PLR elements associated with various central nervous system (CNS) conditions.
Analyze different PLR patterns (elements) in a sample to identify individual conditions.

Advantages over existing laboratory processes and existing PLR devices include:
Speed of result return vs lab.
Portability vs both.
Non-invasive nature vs lab.
Contact with subject not required vs both.
Potential range of determinations of factors affecting CNS vs both.
Lower initial purchase and setup cost per device vs both.
Ease of incorporating improvements into system vs both.
Accuracy vs lab (existing devices do not deliver any result or diagnosis).

It is a feature of the disclosure below that analysis of the fine details of the PLR is accomplished by computer analysis using machine learning. To uncover potential information about functioning of the brain in various disease states and under the influence of chemical substances that alter mood, mental processes or level of consciousness, the methods disclosed herein use a smart phone (or similar Personal Electronic Device (PED)), available to physicians, nurses, and Emergency Medical Technicians (EMT) in the field to upload PLR information to an artificial intelligence network providing a new level of analysis sufficiently detailed to reveal relevant brain functions and/or state.

The present method and system analyzes the PLR in milliseconds and returns the results to the user, providing an extremely timely result in clinical settings and also in the field (e.g. by an EMT). Thus, the methods described herein provide a novel and substantial improvement on previous methods of capturing and/or analyzing the PLR.

Disclosed herein are non-invasive methods, implemented on a personal electronic device, for determining a pupillary light reflex in a subject, including the steps of: (a) providing a light source and exposing one or both pupils of a subject to a flash of light from the light source; (b) capturing one or more videos including the pupil or pupils by a video capturing means; (c) processing image data from the one or more videos so as to extract pupil measurements as a function of time from the image data; and (d) determining one or more PLRs based on the pupil measurements.

In one embodiment, parameters for the flash of light are pre-set in the PED or adjusted manually or adjusted automatically. In one embodiment, the parameters are selected from the group consisting of wavelength, pattern, duration, frequency and distance from eye. In one embodiment, the spectrum of the wavelength of the flash of light is in the visible light spectrum (nominally from 400 nanometers to 700 nanometers). In another embodiment, the spectrum of the wavelength of the flash of light is in the infrared spectrum (nominally from 700 nanometers to 1 millimeter). In one embodiment, the spectrum of the wavelength of the flash of light is about 450 nanometers. In one embodiment, the pattern of the flash of light comprises a spectrum associated with any light emitting diode (LED), multiple flashes, or, in the alternative, no flashes (just the ambient light in the room). In one embodiment, the multiple flashes are continuous, random, or repeating as to flash duration and duration of time between flash illuminations. In one embodiment, the duration of the flash of light is from about 100 milliseconds to about 2000 milliseconds. In one embodiment, the frequency of the flash of light is from about 0.2 Hz to about 4 Hz.

In one embodiment, the light source is spaced from the pupil by about 2 inches to about 8 inches. In one embodiment, the capturing of one or more videos is conducted simultaneously for both pupils. In one embodiment, the capturing is conducted at a frequency of from about 15 frames per second to about 60 frames per second. In one embodiment, capturing is conducted for a period of about 4.5 seconds, or any period between 500 milliseconds and 6000 milliseconds. In one embodiment, the capturing comprises collecting about 30 frames to 270 frames for each video. In one embodiment, the processing is carried out using feature extraction software.

In one embodiment, the steps of providing a light source and capturing one or more videos are carried out using parameters that are pre-set on the PED or using parameters that are adjusted manually or automatically. In one embodiment, the adjusting is carried out based on real-time video capture results.

In one embodiment, the PED is spaced from the pupil by about 2 inches to about 8 inches and nominally spaced from the pupil by about 3 inches. In one embodiment, the PED is selected from the group consisting of: smartphone, tablet, digital camera or computer. In one embodiment, the PED is a smartphone.

In one embodiment, the PLR is used to diagnose a neurological or psychiatric brain condition. In one embodiment, the PLR is used to determine a level of one or more chemical substances in the subject.

Disclosed herein are systems and methods for determining a level of one or more chemical substances in a subject, including the steps of: (a) capturing a plurality of images of one or both of a subject's pupils on a device capable of capturing multiple images over several seconds; (b) extracting pupil measurements as a function of time from the plurality of images so as to determine one or more PLRs; and (c) analyzing the PLR so as to provide a diagnostic output that identifies the presence or absence of one or more chemical substances in the subject.

In one embodiment, the plurality of images comprises more than 100 images captured within 3 seconds, 300 images in 9 seconds or any multiple thereof. In one embodiment, the plurality comprises equally spaced images. In one embodiment, the capturing is carried out using a non-invasive method. In one embodiment, the steps extracting or analyzing or both are preformed on a server. In one embodiment, the server is a cloud-based server.

In one embodiment, the non-invasive method is any non-invasive method described herein that does not involve physical contact with a subject's body. In one embodiment, the capturing comprises exposing the pupils to a flash of light. In one embodiment, the analyzing is carried out using an artificial intelligence network. In one embodiment, the artificial intelligence network is capable of enhanced accuracy of diagnostic outputs by data transmissions to the network. In one embodiment, the method further includes after the step of analyzing, providing feedback data to the artificial intelligence network. In one embodiment, the providing comprises a solicitation as to accuracy of the diagnostic output. In one embodiment, one or more steps of the method are implemented on the device. In one embodiment, the device comprises a camera and a light source. In one embodiment, the device is a personal electronic device.

In one embodiment, the output is transmitted to the device. In one embodiment, the output comprises a format selected from the group consisting of interactive, text, standard assay format, verbal and a graph. In one embodiment, the chemical substances are selected from the group consisting of alcohol, stimulants, neuroleptics, opioids, nicotine, caffeine, phencyclidine (PCP), lysergic acid diethylamide (LSD), dextroamphetamine (Dexedrine-Amedra Pharmaceuticals LLC, Horsham, Pa.), amphetamine, gabapentin, narcotics or any combination thereof. In one embodiment, the diagnostic output comprises the absence of chemical substances.

In one embodiment, the method further includes a context sensitive mode having background probabilities for chemical substances, as described herein.

### BRIEF DESCRIPTION OF THE DRAWINGS

FIG. 1 is a stylized image of a human eye.

FIG. 2 is a graph depicting a sample PLR.

FIG. 3 shows a flowchart of one embodiment of the methods disclosed herein.

FIG. 4 is a screenshot of a subject's eyes being captured by an embodiment of the methods described herein.

FIG. 5 illustrates calibration of a video capture component.

FIG. 6 illustrates feature extraction to facilitate distinguishing a boundary between a pupil and an iris.

FIG. 7 schematically illustrates use of a neural network to input pupillary measurements and output substance identification.

FIG. 8 is a graph depicting a PLR showing a concentration of an amphetamine.

FIG. 9 is a graph depicting a PLR showing a concentration of an opioid.

FIG. 10 shows an example of a diagnostic output of an embodiment of the methods described herein.

FIG. 11 shows an example of a screen shot of a clinician feedback form of an embodiment of the methods described herein.

The subject matter that is regarded as the invention is particularly pointed out and distinctly claimed in the claims at the conclusion of the specification. The foregoing and other objects, features, and advantages of the invention will be apparent from the following detailed description taken in conjunction with the accompanying drawings.

### DETAILED DESCRIPTION

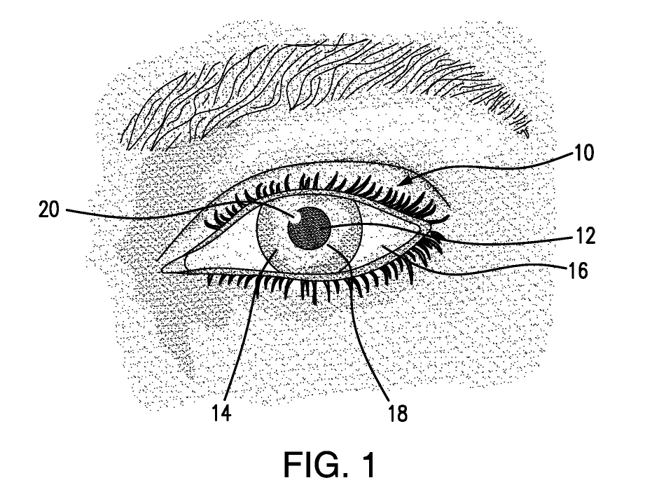

FIG. 1 is a stylized image of a human eye. The eyeball 10 includes a pupil 12, iris 14 and sclera 16. There is a boundary 18 between the pupil 12 and iris 14. While the boundary is easily detected in a person with light colored irises, such as blue or green, the boundary is more difficult to discern for persons with darker colored irises. Because the surface of the eyeball 10 is moist, shadows and reflections 20 are visible and may obscure the boundary 18. For an accurate measurement of the diameter of the pupil 20, the system and method discussed herein solves the technical problem of accurately discerning the boundary 18 when that boundary is in part or entirely obscured by a dark colored iris 14 or shadows and reflections 20.

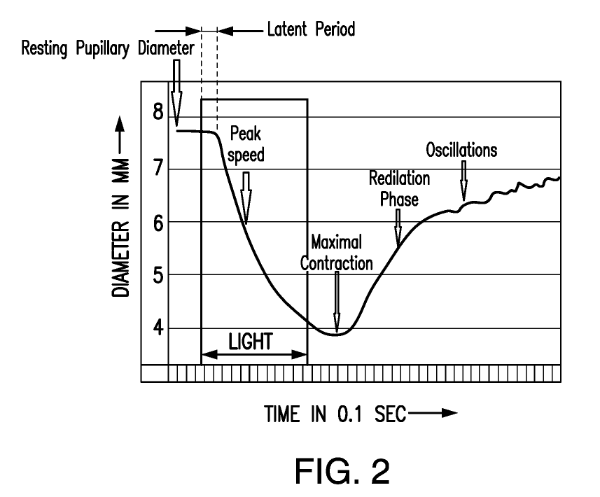

FIG. 2 is a graph depicting a sample PLR in response to a flash of light having a duration of approximately 1.2 seconds. Prior to the flash, the pupil has a resting pupillary diameter that is a function of ambient light. An exemplary resting pupillary diameter is 7.75 millimeters. When the flash is initiated, there is a latent period of approximately 0.15 seconds before a contraction phase begins. The pupil then begins to contract at peak speed for the duration of the flash and a period of time (nominally 0.3 seconds) thereafter to a point of maximal contraction, representing minimum pupil size. Recovery begins with a period of redilation, that is initially relatively constant (redilation phase) which digresses to a series of oscillations as return to the resting pupillary diameter is approached. Both the slope (rate of contraction and rate of dilation) and shape of the curve (oscillations and inflection points) are potentially indicative of factors affecting the central nervous system (CNS), such as a neurological condition of the presence of a chemical substance.

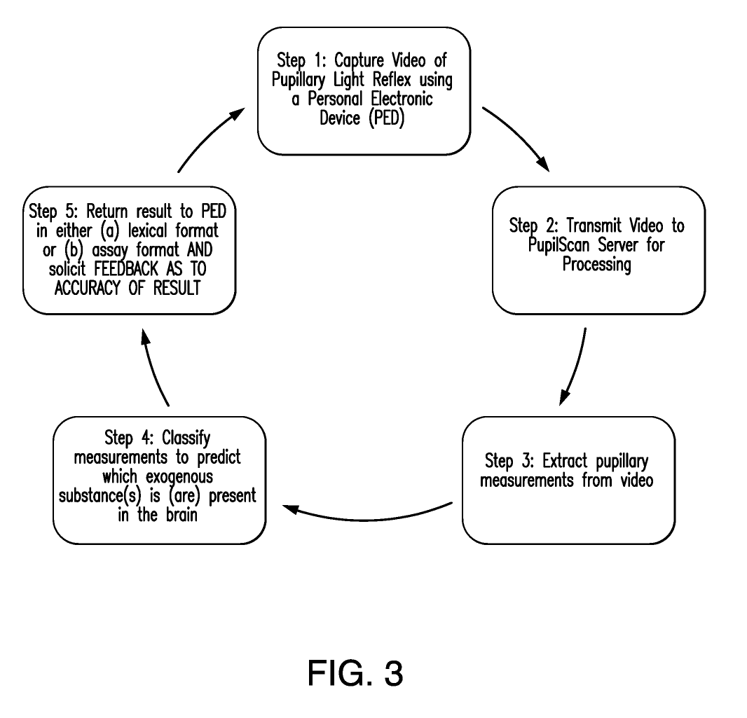

FIG. 3 is a flow chart illustrating a sequence of steps effective to obtain a PLR and then utilize the PLR as a tool to diagnose and treat detected conditions, diseases and disorders. Each step is described in further detail herein below. In Step 1, a video of the PLR is captured by a medical personnel or first responder, preferably on a personal electronic device. In Step 2, the video is transmitted to a server for processing. In Step 3, video processing extracts pupillary measurements from the video. In Step 4, the pupillary measurements are utilized to predict exogenous substances present in the brain. In Step 5, the results are returned to the medical personnel of first responder in either lexical format or assay format. Optionally, following Step 5, the recipient may provide feedback as to the accuracy of the result to facilitate improved accuracy by way of machine learning.

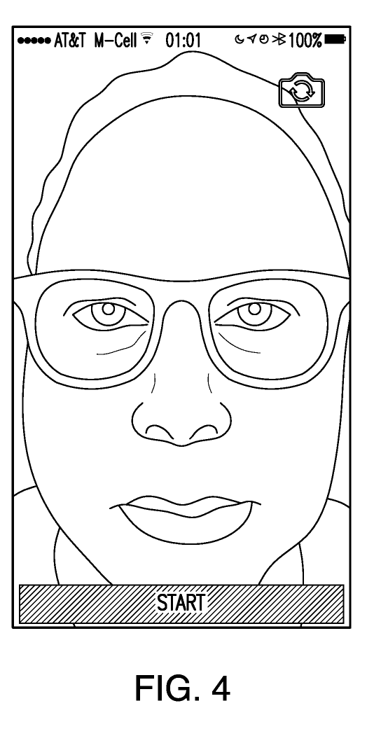

Step 1: A user holds a light source and video capture device, preferably a PED, at a distance from a test subject's eyes. The distance is preferably the minimum distance that captures both eyeballs in the same frame. It is desirable to be as close as possible to the eyeballs, without touching the subject, to maximize the resolution to facilitate measurement of pupil diameter. It is desirable to capture both eyeballs in the same frame because absent severe neurological damage, the responses of both eyeballs are essentially the same. Having two eyeballs in the frame enables selection of the one having better resolution. The video capture is non-invasive, neither the light source nor the recording apparatus contacts the test subject. For a smart phone the distance between the smart phone and the test subject is between 2 inches and 8 inches, and nominally 3 inches. The method and the system disclosed herein require no contact with the patient and do not require the patient to be conscious (an ability to test a non-responsive subject is discussed below). Any methodology that captures at least one pupil (possibly including surrounding ophthalmic and facial tissue) is sufficient for analysis. An exemplary screenshot of a user's eyes being captured by a smartphone application is shown in FIG. 4.

The methods described herein for capturing the pupillary light reflex and eye movement preferably utilize a smartphone or other handheld device. The device has a video capturing component, for example, a high-resolution camera, and a light source, for example, a flash. The video capturing component encompasses a camera or other device capable of collecting a plurality of evenly spaced images at a frequency of at least 5 frames per second. Furthermore, the disclosed methods enable the capture of PLR without the device coming in contact with the patient thus enabling the PLR to be captured non-invasively. In some embodiments, one or more lasers or other non-contact method is used to determine pupil configuration, shape and/or size. As used herein, “pupillary light reflex” means the change in the size of the pupil (diameter, circumference, area, other measurable parameters) over time before and after the pupil is exposed to a flash of light. Single or multiple PLRs may be collected and analyzed as part of the same subject study, either in one video sequence or in separate sequences.

The light source is any source capable of emitting a flash of light at various wavelengths, patterns, duration, frequency and distances from eye. In some embodiments, such parameters of the flash of light may be preset, determined manually at time of sample collection, or determined automatically by the system based on real time PLR results and other indicators including, but not limited to, ambient light, subject light factors such as eye coloring, and others. Automatic flash adjustments may be made and applied within the context of one video sample. Adjustments may also be made based on a sample then applied to a subsequent sample, or made based on existing conditions then applied to a sample when collected in those conditions.

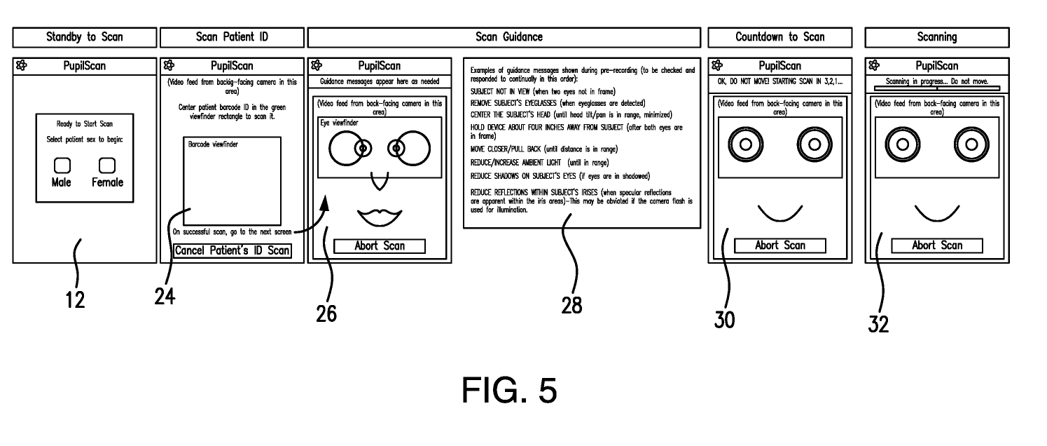

High quality scans (videos) for analysis by the back-end server and a process to obtain those videos with metadata attached is described with reference to FIG. 5. Including metadata enables the back-end server to do a better job processing images. Pupil diameter measurements are made at the handheld device and transferred to the back-end server as metadata appended to the frame. This embodiment enables relatively advanced image analysis in real time at the handheld device by guiding a clinician to take an optimal scan and then provides metadata so that the back-end processing can more easily locate and measure the pupil, the iris, and other features/movements of the eye. When the system is running, it is identifying
(a) The location of the center of the pupil in each frame;
(b) Movements of the center of the pupil from frame to frame for the purpose of calculating eye movements that will be used to augment the accuracy of the conclusions drawn from the PLR alone;
(c) Background light conditions and reflections off the pupil, iris, and cornea; with real-time guidance to the clinician about how to move the PED so as to eliminate negative conditions. The guidance may be visual, verbal, and/or tactile. Such guidance may advise regarding too far away, too close, reflections, insufficient background light, shadows that cover key parts of the video, etc.; and
(d) Boundary drawings (image segmentation) in real-time that “find” the general location of the iris and the pupil such that these x,y coordinates (and in some case full mappings) are reported back to the server with each frame of video.

Many factors impact the ability of handheld device software to obtain accurate measurements. Some are a function of the environment (light levels, light directions, light colors, light sources, light reflections, shadows, motion blur and occlusion). Some are inherent in the data (iris color). The PLR creates an inherent dilemma in the data-gathering process: a certain amount of light is needed to take images, but light in any amount affects pupillary diameter. An ideal recording features soft, even lighting, no specular reflections in the eye, no shadows on the face, eye sockets not shadowed, eyes open and not occluded and looking ahead, no eyeglasses, face not tilted or panned, and a proper distance maintained throughout recording between the subject and camera. An ideal subject has light-colored irises, making pupil segmentation easier, and falls in the middle of the distribution curves for interpupillary distance (IPD) and corneal width.

This software guides the clinician-user to obtain the best possible video quality by adjusting positioning of the device, lighting, and other factors. Guidance is automated and presented on-screen (with a verbal delivery option). Only after various conditions are within tolerance ranges will the software automatically begin a countdown to recording. Conditions must remain in range throughout the recording period; if they go out of range, the recording is stopped and the user informed. Any collected data should be sent to the server in any case.

The system has two technical components: 1) the client side (the software that runs on smartphone platforms), and 2) the server side (also referred to herein as the remote processor). The smartphone platform includes functionality supported by both iOS (Apple, Inc., Cupertino, Calif.) and Android (Google LLC, Mountain View, Calif.). One feature is an open source eye-tracking software package such as Drishti (Drishti Technologies, Inc., Palo Alto, Calif.). Drishti provides, for each eye in each frame of a video, 27 eye positioning points. Running Drishti, or similar software, on the client side provides real-time positional guidance to the user before and during the recording process. In addition to software user guidance and condition monitoring, the eye-points data are also very helpful for informing iris and pupil segmentation on the server side. There is no need to run Drishti again on the server side because the data will be provided by the device. Rather than Drishti, any image segmentation system designed specifically for the eye/face which returns eye-positioning points and boundary data may be utilized.

It is preferred that many settings are able to be remotely configurable such that when a setting is changed on the server side, all installations of the software will use the new setting. One example would be the frames per second (FPS) rate of the video during capture. Other such settings could include anything to do with software user guidance (see below), such as the minimum amount of ambient light required, or the threshold for dark irises.

The back end, server side, platform n includes a MATLAB cluster for running the image processing and MATLAB components of the project. It could also include a Linux box. Both run behind a secure firewall. The Linux box contains a database to store app and user registration info and software settings, including calibration. It hosts scripts needed for providing information to the software (e.g., when the software checks for current global settings before each scan) and accepting information from it (e.g., when a new device or user is registered).

Prior to first use, the device is registered, calibrated and settings installed. Calibration is optional when size assumptions are made based on average corneal width and inter-pupillary distance. The app cannot take scans unless: 1) App is registered, 2) a registered user is logged in, and 3) app is calibrated. Each app installation should have its own ID and profile in the server-side database. Among the information collected and saved is information about the device itself including platform, operating system and version, users who have logged in to the device, when app was registered, version of the app that is running.

Referencing FIG. 5, once the device is registered, a registered user is logged in, and app has been calibrated, the device will set to “Standby to Scan” mode 22, which is the first screen of the scan procedure. A first, optional, step may be to identify the subject as male (M) or female (F) to assist with inter-pupillary distance (IPD) calibration. IPD is relatively constant from person to person. For an adult mail, the IPD is typically around 140 millimeters and for an adult female, the IPD is around 132 millimeters. Sex is also useful to predict iris dimensions to obtain a pixel to millimeter ratio.

Selection advances to patient identification screen 24. If the patient is in the hospital, the patient's identification bracelet may be scanned. If the patient is conscious or is identified, the patient's name or other identification is entered into the system. This enables a search of the system database for previous pupillary scans or other relevant medical history of the patient. If no identification of the patient is pre-existing, a new identification code is generated.

The system then advances to a guidance phase 26 to optimize positioning, lighting and other factors. Among the factors considered 28 are acceptable ranges for: eyeglasses off;

ambient light level;

visibility of eyes good;

apparent size of eye features (distance change proxy);

shadows

head tilt/pan

specular reflections

eye blink count

A countdown phase 30 automatically begins when the image is within tolerances. At the end of the countdown, scanning phase 32 commences. An exemplary recording period includes: 500 ms of baseline video, 1000 ms light stimulus via camera flash, then 4500 ms of recording. The FPS rate is determined by making a request for the current settings from the server. The system monitors for changes in positioning/lighting/distance/occlusion/etc. and stops scanning if a parameter falls outside predefined tolerances. When the scan is completed, the data is verified, for example if the number of eye blinks in the video exceeds a predefined tolerances, such as 20% of the frames. As the pupil diameter cannot be measured when the eye is closed, excessive blinking leads to a loss of resolution. The verified data is then compiled and uploaded to the server. The device then returns to standby to scan screen 22.

During scan guidance 28, the app ensures that there is adequate illumination, and also will not perform the scan (or will make some audible objection) if there are problems with illumination, namely (a) reflections that occur within an area of interest (the inner 80% of the iris and the boundaries between the iris and the pupil); (b) shadows that occur on the iris; and (c) a check that the general “size” in pixels of the images of the pupils and irises are sufficient for measurement. Measurements may be up-loaded to the backend server in pixels or converted from pixels to millimeters.

One challenge is to measure pupillary diameter in the absence of any markers of known size in the images and an unknown distance between the camera and subject. One option is to use human facial features that have low standard deviation to calibrate the app in order to estimate a pixel-to-nun ratio for measuring within scan images. Exemplary features with low standard deviation are inter-pupillary distance (IPD) and corneal width. Consider that the IPD as measured will change depending on where the eyes are focused, which could introduce error. IPD measurement might best be done from a greater distance than the rest of the pupil scan to encourage a deeper focus and a more centered gaze. This would require an additional separate step in the scanning process.

A second option is to affix a marker, such as a one centimeter diameter disc, on the bridge of the test subject's nose as a reference indicia.

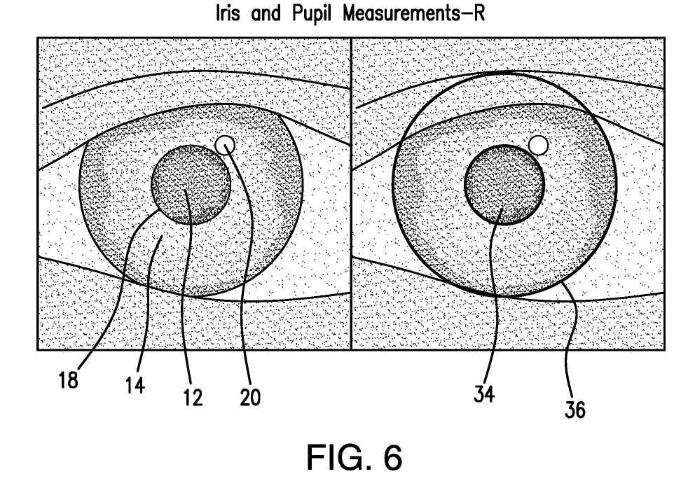

Referencing FIG. 6, for subjects with dark color irises 14, it is frequently difficult to discern the boundary 18 between the iris and the pupil 12 making pupil diameter measurement difficult. Also, a shadow or reflection 20 may overlap the boundary 18. Image extraction software is included in the handheld device so that images of the pupils 34 and images of the iris 36 are first extracted using standard extraction software on the device. The Drishti iris center location may be used to center a 150 pixel by 150 pixel region to be extract from each frame. Feature extraction requires substantial memory and processing capability. Preferably, feature extraction is not applied to every video frame, rather periodically. Feature extraction is applied to every “nth” frame where “n” is an integer greater than 1. For example, when n equals 4, feature extraction is applied to each fourth frame. Once the pupils are extracted from a frame, their lateral diameter is measured. The measurements may be in pixels or the handheld device may contain software to convert pixels to millimeters. Other measures of pupil size may be extracted, such as total pupil area and pupil to iris ratio (either in area or in diameter).

Exemplary video capture is 135 frames of a 4.5 second video (See Guyon, Gunn, Nikravesh, Zadeh, eds, Studies in Fuzziness and Soft Computing, (Springer 2006), ISBN 978-3540354871) resulting in 270 data points available to be passed to the backend server (2 pupils×135 frames). Lateral diameter measurements are appended to the metadata of every frame, such as every fourth frame, with feature extraction processing.

Alcohol is present, but not the primary toxin, in 67% of ED admissions that require toxicological analysis in the U.S. Nystagmus is a reliable indicator of alcohol intoxication. Nystagmus is characterized by the gaze drifting away slowly from its point of focus (slow phase) and then snapping back quickly (fast phase). These quick phases of nystagmus are rapid, with maximum velocities as high as 500 degrees per second. These rapid eye movements are evolutionary forerunners of voluntary saccades. It is preferable for the backend server to have a separate vector for alcohol, to complement the pupillary measurements and help discriminate between substances. Therefore, in addition to pupillary diameter, some embodiments of the disclosed system and device also track and measure eye movements including, but not limited to, smooth pursuits, saccades, visual fixation movements, vergences and the various types of nystagmus.

Step 2: In this instantiation, the video captured in Step 1 is transmitted, preferably by wireless data communication, to a server. The video may be transmitted from any PED by any means to a server, or may be processed or pre-processed on the PED itself. The light flashes may be changed, either at the PED or remotely. The video can alternatively be transmitted by wired data communication or physically (e.g. by using a universal serial bus (USB) stick). The video can be captured by any device capable of rendering multiple images of the pupil over several seconds.

Following capture, the video of the subject's eyes is then transmitted (for example, by wireless data connection in the case of the smartphone) to a server. The server is programmed to perform at least the following two functions: (1) feature extraction/measurement from the iris images where such did not occur at the handheld device, and (2) PLR recognition and classification. In some embodiments, the server is a cloud-based server. In some embodiments, native analysis of PLR (all or part of analytical process) is performed locally on the device without transmission of some or all data to a remote server or analytic engine.

The data package sent to the server at the end of the scan/capture includes:

1) User/patient/app data for scan: app ID, user ID, patient ID from wristband;

2) Video file of scan period;

3) Baseline data for scan: timestamp, latitude/longitude coordinates, patient sex, iris color, ambient light level, distance estimate, remotely-configured settings (FPS rate, minimum ambient light, dark iris threshold, etc.); and

4) Per frame data: ambient light level, distance estimate, location point data for each eye, whether each eye is open or not.

Step 3: Images of the pupils are extracted from the video of the subject's eyes. In one embodiment, the video captures 135 frames in 4.5 seconds, resulting in 270 measurements total (2 pupils×135 frames) taken about 33 milliseconds apart. In other embodiments there are adjustable parameters, for example, the duration, flash timing, and frequency of frame capture are all adjustable parameters of the methods disclosed herein and can be adapted as further described herein (adaptive parameter changes). These measurements (or similar measurements), taken as a time series, constitute the PLR. Measurements may be taken from extracted images of the iris and the pupil. In one embodiment, the method further includes analysis of micro-oscillations which may yield information on identification of factors affecting CNS.

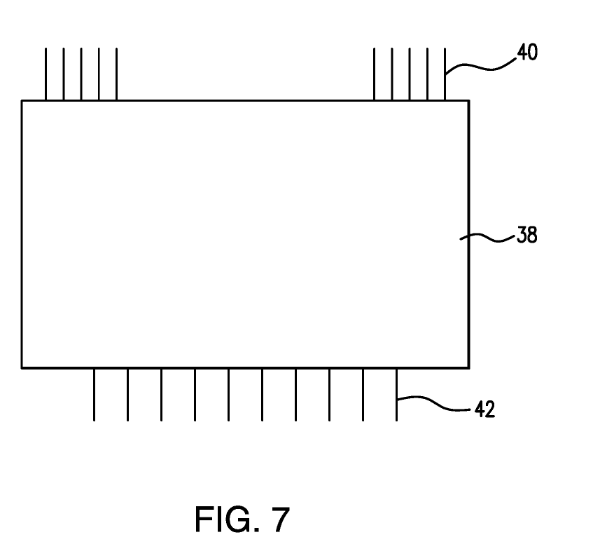

Step 4: FIG. 7 schematically illustrates a neural network 38 where the 270 measurements are used as inputs into 270 input nodes 40 of a multi-layer “deep learning” back-propagation neural network that has output nodes 42 corresponding to specific substances and substance types, and hidden layers suitable to support a convolutional neural network (such as that developed and published by Alex Krizhevsky at the 2012 Neural Information and Processing Systems Conference in Lake Tahoe, Nev., “ImageNet Classification with Deep Convolutional Neural Networks”, published in NIPS Conference Proceedings on Nov. 17, 2013).

The present embodiments may use either a convolutional model or non-convolutional model. Of note, the convolutional model obviates Step 3, and is more accurate than the non-convolutional model, but requires higher quality data input. Many other proprietary and non-proprietary classification tools are used for Step 4, always competing for accuracy with the multi-layer deep-learning back-propagation neural network. These methods include, but are not limited to, Support Vector Machines, Graph-Theory-Based Classification Algorithms, and Feed-Forward (unsupervised) learning networks.

Convolutional methods obviate Step 3. In the event that convolutional methods are used, the raw input to the server is the video itself, not the measurements. Non-convolutional methods require that the pupil measurements first be extracted from the video. Convolutional methods incorporate that extraction implicitly and thus use the video itself as input, not the measurements of the pupil.

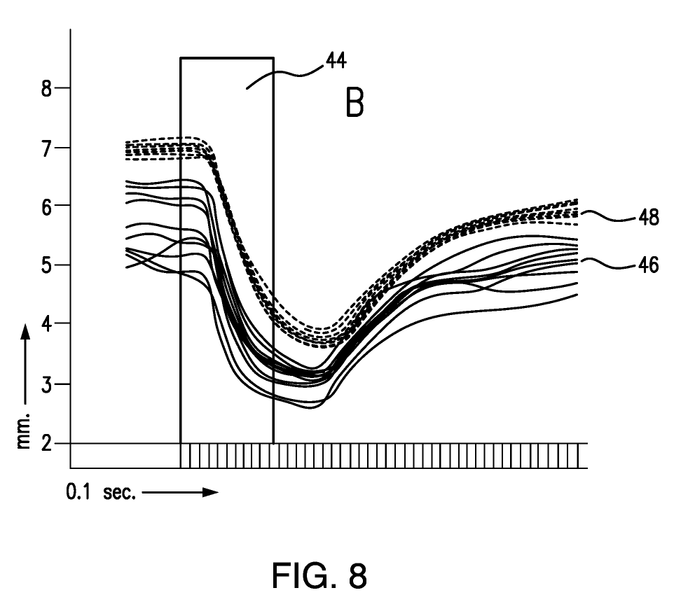

After the PLR is extracted, the PLR is analyzed for patterns using a host of machine learning techniques. FIG. 8 shows one such pattern for an amphetamine, benzedrine. Pupil diameter was measured as a function of time following a flash of light having a pulse duration 44 of one second. The solid lines 46 are test subjects who had not ingested the drug. The dashed lines 48 are test subjects who had ingested 10 mg of benzedrine one hour before the test. The benzedrine induced pupillary response shows a more pronounced redilation and less oscillations.

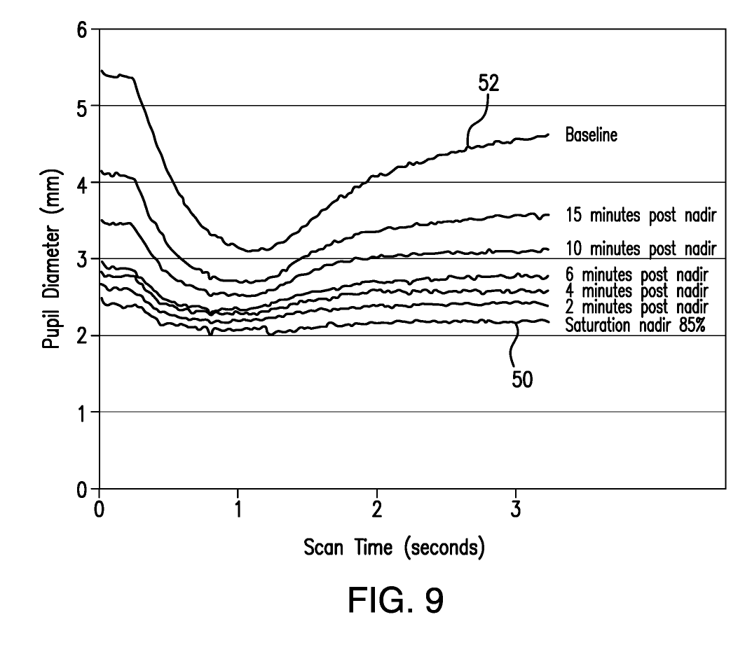

FIG. 9 shows one such pattern for an opioid. At near overdose levels 50 (blood oxygen saturation level at 85% rather than close to 100%), the pupils are contracted prior to a light flash compared to baseline 52 (no opioid ingested) pupil diameter and there is virtually no redilation.

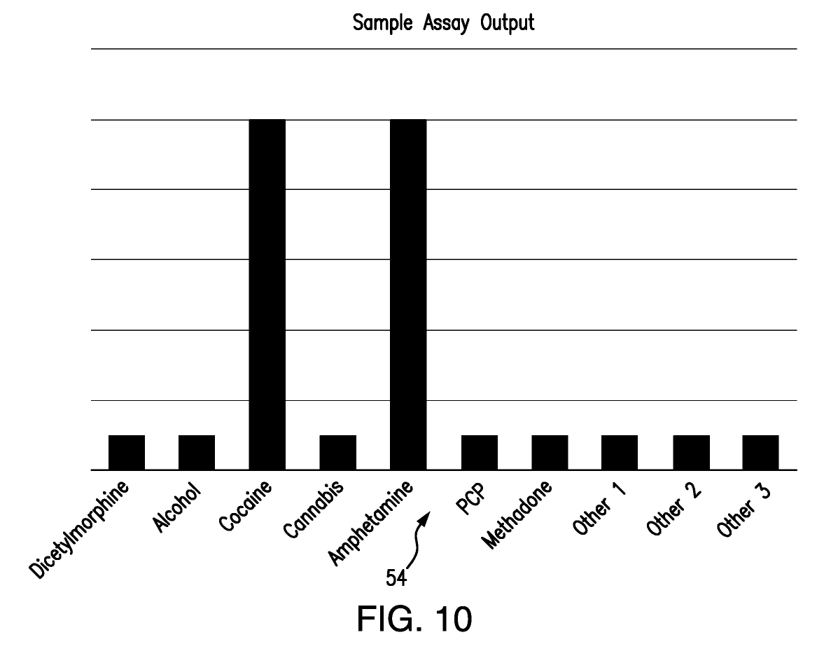

Step 5: In some embodiments, the server then returns results of the analysis directly to the handheld device. In one embodiment, FIG. 10, the results are toxicological and identify psychotropic substances 54 likely present in the subject's brain. Each psychotropic substance 54 corresponds to an output node 42 (FIG. 7) from the neural network. The higher the output, the more likely the substance is present. FIG. 10 is exemplary for a mixture of cocaine and an amphetamine. Other embodiments identify other brain states covering indications across neurology and psychiatry, for example seizure propensity or major depressive disorder.

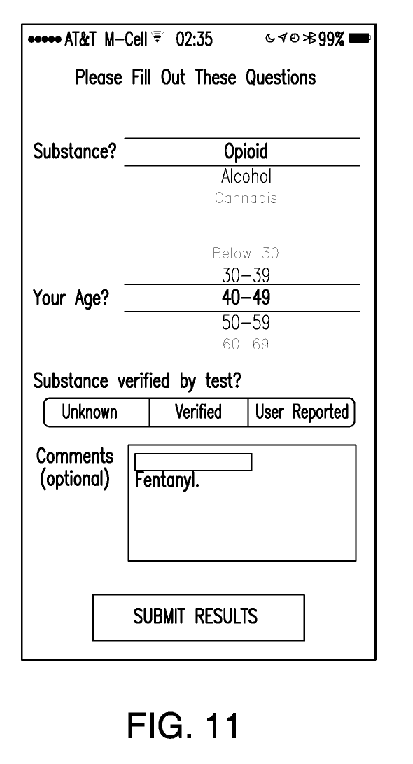

The results may be returned to the clinician on the PED in a variety of formats, for example:
a) A list of substance(s) detected by Step 4 (see FIG. 11) or other textual description.
b) An assay-style result (see FIG. 10) that mimics those produced by standard blood analysis assay tests and toxicological screen. A bar graph or other format displays substances with high and low probability of significant presence. The graphical display has advantages including:
Similarity to result format in current use for presentation of laboratory results.
My display on same device used to collect sample(s), or may be displayed elsewhere or transmitted by other method(s).
Other substances and/or indications may be included in output, and probabilities may vary both in relative and absolute terms.

Steps 1-5 are a continuously recurring cycle whereby the system learns as it operates, by getting feedback pertaining to the results returned to the clinician (see FIG. 11), for example:
a) Clinician intuition: Clinician may agree or disagree with the results, and state his or her opinion as to the “true” toxin(s)
) Patient reported toxin: Patient may state a particular toxin or toxin(s)
c) Results of fluid-based tests (for example antibody assays or mass spectrometry) conducted on the patient can be used to “Verify” the output.
d) As part of Step 5, this “feedback” data is returned to the server so that the neural network can be modified to increase accuracy. The present invention thereby incorporates a virtuous cycle such that its accuracy improves over time. To further explain, the neural network is trained in two steps:
   a. Non-use training-only feedback. In this step, no result is produced. Instead, the neural network is trained with the results of Step 3 as input, and a fluid-based test result as the training set data. When convolutional neural networks are used, Step 3 is obviated and the video itself is input, and the fluid-based test result is output. Once this training achieves a sufficient level of sensitivity, specificity, positive predictive value (PPV) and negative predictive value (NPV) it is deployed clinically.
   b. However, those four measures of accuracy (sensitivity, specificity, PPV and NPV) continue to be improved upon by asking the clinician to fill out a form to improve the classification model's accuracy:
      i. When a clinician elects to fill out the “feedback form” on the results (see, for example, FIG. 6), these are automatically incorporated into improving the model, using nonmonotonic defeasible reasoning to decide which feedback should be propagated through the network and how. For example, intuitive feedback from a historically unreliable source (clinician or patient) is discounted, while “verified” test results contradicting the results are given a much higher weight.

In rare cases, manual examination is required by data scientists and/or ophthalmologists working together to improve the neural network model accuracy. However, the invention improves its accuracy, by itself, the more it is used in clinical practice.

The method disclosed herein may offer a “context sensitive mode” or CSM.

There are two sub-modes of this: automatic and manual. If the user sets CSM to on, the software determines context geosymantically, unless the user selects from a multiple choice list the intended clinical use of the software, e.g. “Emergency Department (ED)” or “EMT Field Use”. This is important because the background probabilities for the presence of toxins are very different in different use cases. For example, the background probability (knowing nothing else) of diacetylmorphine presence in a toxin screen is much higher in an EMT setting than in an ED setting. If CSM is switched on, defeasible reasoning (see, See Pollock, J L, Hosea, D F, “An Artificially Intelligent Advisor for Emergency Room Medicine”, Proceedings of AI in Medicine Conference, Stanford University, April 1996, published October 1997) is deployed in Step 4 to adjust for these background probabilities, returning results that take the context into consideration. CSM may be used at the option of the medical caregiver, but offers an advantage because it is well documented that medical caregivers sometimes fail to take into account background probabilities.

Eye movements, including, but not limited to the pupil phenomena, are closely linked to states of ANS and CNS functional abnormality (intoxication) and furthermore to various substances. Since we now have eye-movements data from the client-side software, the server-side software looks at both the PLR and eye movements. Similar to the above-analysis of the pupillary light reflex, the system analyses those reflexes that evolved to stabilize images on the retina in particular during head perturbations:

Vestibular Ocular Reflexes and Visually Mediated Reflexes (optokinetic and smooth pursuit tracking)—are two distinct mechanisms termed gaze stabilizing reflexes. These gaze shifting reflexes keep the fovea of each eye pointed at the object of regard whenever the head is moving.

There is also a repertoire of gaze-shifting movements. These are necessary to change the line of sight independently of head movements. With the evolution of the fovea, a central area of the retina with maximum visual sensitivity, it became important to be able to direct this specialized area of the retina at the object of interest during visual search or the appearance of new information in the visual periphery. This redirection of the line of sight is termed a saccade. They are generated under a broad range of conditions. For example, a saccade may be triggered by the appearance novel objects seen or heard, voluntarily during visual search, from memory, or as part of a learned motor behavior. There is usually a delay of about 200 ms from the stimulus for a saccade until its enactment, and this time includes neural processing in the retina, cerebral cortex, superior colliculus, basal ganglia, thalamus, and cerebellum. The final neural command for voluntary saccades arises from the same brainstem neurons in the paramedian reticular formation that generate the quick phases of nystagmus. Normal saccades are fast, brief and accurate. Disease, or the influence of drugs, may cause them to become slow, prolonged or inaccurate when they may cause visual disability and be measurable. With the evolution of the fovea also came the need to track a moving object smoothly. Pursuit allows us to maintain the image of a smoothly moving object close to the fovea. Saccades may capture the image of a moving target on the fovea, but, without pursuit, the image soon slides off again, with a consequent decline in visual acuity. Smooth pursuit performance is impaired with cerebellar disease and is susceptible to many drugs with effects on the nervous system. Holding an image of a stationary object on the fovea by minimizing ocular drifts is also a class of eye movements termed Visual Fixation. There are several types of fixational eye movements including microsaccades, slow-control, field holding reflex, and the ocular following response. Also important, with the evolution of frontal vision and binocularity, disjunctive or vergence eye movements became necessary so that images of an object of interest could be placed on the fovea of each eye simultaneously and then held there. Thus, eye movements are of two main types: those that stabilize gaze and in so doing keep image steady on the retina and those that shift gaze and in so doing redirect the line of sight to a new object of interest. Each functional class of eye movements is linked to anatomical circuits in and from the brain to the eye. Analysis of these movements provides insights regarding specific disease and toxicology influences on the brain that direct these eye movements

Functional classes of eye movements are described in Table 1 below:

TABLE 1
FUNCTIONAL CLASSES OF HUMAN EYE MOVEMENTS

| Class of Eye Movement | Main Function |
|---|---|
| Vestibular | Holds images of the visual world steady on |
| | the retina during brief head rotations or |
| | translations |
| Visual Fixation | Holds the image of a stationary object on |
| | the fovea by minimizing ocular drifts |
| Optokinetic | Holds images of the visual world steady on |
| | the retina during sustained head rotation or |
| | translations |
| Smooth Pursuit | Holds the image of a small moving target |
| | on the fovea; or holds the image of a small |
| | target on the retina that is close to the head, |
| | during linear motion (translation); with |
| | optokinetic responses, aids gaze |
| | stabilization during sustained head rotation |
| Nystagmus | Reset the eyes during prolonged rotation |
| | and direct gaze toward the oncoming visual |
| | scene |
| Saccades | Brings images of object of interest onto the |
| | fovea |
| Vergence | Moves the eyes in opposite directions so |
| | that images of a single object are placed or |
| | held simultaneously on the fovea of each |
| | eye |

Testing each of these eye movements in isolation will help identify specific defects, caused by diseases, injury, drugs and other causes, that will be useful in diagnostic localization and treatment. The abnormalities of these eye movements are distinctive and will point to specific pathophysiology, anatomical localization, or pharmacological disturbance. These eye movements correspond directly to psychotropic substances in the ANS and CNS (see “The Neurology of Eye Movements”, by R John Leigh (Case Western Reserve) and David S Zee (Johns Hopkins), 2015). By combining the PLR analysis with the eye movement analysis the system may produce substantially more accurate results in toxicology. Furthermore, the system may produce much better results in polysubstance toxicology. An analysis combining PLR with eye movement is much more powerful than either alone. The methods disclosed herein and similar analytical techniques may be used to discern many other neurophysiological, neurological, psychiatric, and somatic states, and to produce other analytical outputs such as prognostic outputs and other diagnostic outputs.

There are many more medical indications (beyond substance toxicology) that can be diagnosed from the PLR and/or eye movement analysis. As a non-comprehensive listing following is a list of various diseases with pupillary signs:

Syphilis—distinguish among various stages.

Viral Infections
a. Viral Encephalitis;
b. Herpes Zoster (Shingles);
c. Polio; and
d. Viral Meningitis
e. Viral Childhood Diseases—Varicella (Chicken Pox), Rubella, Measles, Mumps, Pertussis

Bacterial Infections
a. Tuberculosis;
b. Sarcoidosis;
c. Bacterial Encephalitis and Meningoencephalitis; and
d. Other bacterial and fungal diseases that can damage pupilloconstrictor muscles including Hansen's Disease (Leprosy)

Toxin Producing Bacteria
a. Tetanus—mydriasis and oscillations of large amplitude;
b. Botulism—accommodative loss, and paralysis of pupillary sphincter with large fixed pupils and internal ophthalmoplegia; and
c. Diphtheria—Accommodative loss and normal PLR.

Parasitic Infection—Toxic Reaction with pinpoint pupils vs accommodative palsy and mydriasis.

Embolic Infections with Destruction Along Pupillary Pathways

Ear Infections and Surgical Trauma
a. Damage to sympathetic fibers:
   1. As they pass beneath the mucosa of the tympanic capsule;
   2. In their adjoining pericarotid course;
   3. Intracranial, near the end of the Gasserian ganglion where they continue with the division of the ophthalmic division of the trigeminal nerve (Raeder's Syndrome); and
   4. Ptosis and Miosis ipsilateral eye

Infections of the Face and Jaws
a. Abscessed Tooth Causing Horner's Syndrome or the converse of sympathetic stimulation and mydriasis and slight reduction in the light reflex but without accommodative loss or impairment of extraocular movements;
b. Infection in the upper jaw affecting the sympathetic fibers and the ophthalmic branch of the trigeminal nerve; and
c. Impairment of the sympathetic oculo-pupillary fibers and the ophthalmic 5^{th} nerve combined with an oculomotor deficit affecting the superior orbital fissure or the cavernous sinus.

Infections of the Paranasal Sinuses:
a. Sphenoidal Sinusitis and Sphenoidal Mucocele;
b. The Superior Orbital Fissure and Orbital Apex Syndrome Syndromes; and
c. Spread of Infection to the Orbit and Intracranial Venous sinuses.

Thyroid Disease

Sympathetic Deficit: Homer's Syndrome can result when a thyroid mass compresses the cervical sympathetic chain or when the nerve or its blood supply is injured during surgery when a tumor is removed.

Apparent Sympathetic Stimulation in Thyrotoxicosis:

Lid retraction and exophthalmos are not caused by sympathetic stimulation.

Peculiar pupillary “dazzling” syndrome

Rare reaction pattern in ⅓ of patients with thyroid conditions:
Light flashes are unpleasantly bright;
Supernormal B waves on the Electroretinogram.

DIABETES MELLITUS—The most common pupillary pathology is fairly small sluggish pupils which can in some part be accounted for by the patients' age. Smaller pupils than average for the patient's age. About ⅓ of diabetic patients and none younger than 40 years old exhibit sluggish pupils.

The sluggishness is of a particular type. The pupils are not small enough to explain their slow movements by spasticity of the sphincter muscle. The contractions elicited by 1 second or longer light stimuli are fairly extensive, proving that the 3^{rd} nerve nucleus is able to discharge parasympathetic impulses and that these are conducted to the iris sphincter. But the movements are unusually slow and the latent period of the reactions is prolonged, compared to age-related normal subjects. In response to short, repeated light flashes, presented at a rapid rate the pupils can follow only poorly. While normal pupils, (except in extreme old age) can oscillate in response to 3-per-second light flashes, the diabetic “sticky” pupils cannot follow at 2.5 or even slower rates, recording pupil oscillations to sinusoidal stimuli confirmed this sluggishness. Further, the small, rapid “pupillary unrest” normally seen under the influence of steady light is reduced in such eyes

With diabetic 3^{rd} nerve palsy, the pupil is usually not involved as the ischemic insult causing the 3^{rd} nerve dysfunction is diabetic effects at the core of the nerve and the pupillary fibers run superficially close to the surface of the nerve. In a small percentage of diabetic 3^{rd} nerve palsy the pupillary fibers are included in the lesion. Such pupils are relatively large and react feebly (if at all) to physiologic stimuli.

Argyll Robertson or Spastic Miotic Pupils—occasionally found in diabetic patients some of which had additional extensive neuropathy resembling tabes dorsalis (Neurosyphilis) the condition was termed diabetic pseudo-tabes, paresthesias in the lower extremities, shooting pains and loss of vibratory and proprioceptive sense and well as deep tendon reflex abnormalities.

Neurogenic Tonic Pupils—Small dissociated pupils that move with extreme sluggishness. Ischemic damage to the nerve endings in the pupillary sphincter with aberrant regrowth.

Hypoglycemia—Any disease that triggers a response by the autonomic nervous system will be detectable by the system. Hypoglycemia is a common side effect in diabetics who are on medications, it can occur with injected insulin or on oral hypoglycemics, to regulate their serum glucose level. Lacking the autoregulation nondiabetics have, it is common for patients with diabetes to overestimate the need for medication, either too much medication or not enough glucose intake. Current guidelines are for tight control of glucose (studies show it reduces the long-term side effects of diabetes such as eye and kidney disease), so it is increasingly common to overestimate the dose of hypoglycemic medication and cause hypoglycemia. This lack of glucose is not a benign process, it can start with clumsiness, trouble talking and confusion and progress to loss of consciousness, seizures and death. Neuroglycopenia will likely kill a number of brain cells each time it happens, it is an event to be avoided. It can be difficult to detect as the peripheral autonomic effects of hypoglycemia such as sweating, palpitations, tachycardia (fast heart rate), abdominal discomfort, and skin pallor, are not only subtle but can be suppressed by other medications that diabetics are prescribed for control of blood pressure, beta blockers, which block beta adrenergic effects. So the peripheral effects of the hypoglycemia are not detected by the patient or the caregiver and the reaction goes undetected in its early, treatable, and less dangerous phase. However, the system, which will show the PLR and dilated pupil, which are central effects, can be a better way to diagnosis this important complication of diabetic treatment.

The falling glucose does trigger an epinephrine response, which will cause at least a mydriasis of the pupil and likely the same effect on the PLR other stimulants cause, including a loss of oscillations. When hypoglycemia happens, you can measure your glucose level, if you have your glucometer with you, and you do have to stick yourself with a lancet, and if you have the presence of mind and ability to do this, or it can be detected by the system. This is not just for patients with diabetes, but for all medical providers who take care of diabetics including EMT's and airline flight attendants.

CYSTIC FIBROSIS—High incidence of defective consensual light reflexes. Distinct age trend, with increasing age the incidence grew to 70%. A small % of children had unilateral parasympathetic efferent deficit, preganglionic in type, the affected pupil reacted less extensively than the normal fellow pupil to light and to near vision, without tonic features.

AMYLOIDOSIS—Sluggish pupils, likely from iris damage.

DEMENTIA—Age-related loss of pupillary size begins early, immediately after completion of growth and maturation and it progresses linearly during the following decades. The increasing miosis is almost selectively due to a lessening of the central inhibition of the pupilloconstrictor nucleus. Are such changes accentuated in patients with, for example, early onset Alzheimer's disease? No one knows, yet. Nursing home patients with “Organic Brain Syndromes”, unrelated to infections, trauma or strokes did not have a reduction in pupillary size in darkness compared to age-related normals, and did have a less extensive PLR.

PARKINSON'S DISEASE—Post-encephalitic Parkinsonism can have Argyll Robertson pupils and other midbrain syndromes. Idiopathic Parkinsonism does not have notable pupillary pathology. The system may be useful in drug monitoring.

ATAXIAS (Spinocerebellar Degeneration) and Neuropathic and other Muscular S\Dystrophies—Sluggish pupils.

LOWER MOTOR NEURON DISEASE —Distorted miotic pupils, similar to syphilis.

CHRONIC PROGRESSIVE EXTERNAL OPHTHALMOPLEGIA, MYOTONIA CONGENITA, MYASTHENIA GRAVIS, SYRINGOMYELIA and MULTIPLE SCLEROSIS—Afferent defects from optic neuritis.

NARCOLEPSY AND ADHD—Pupil studies may be important in future studies on a variety of sleep disorders. The measurement of spontaneous pupillary oscillations in darkness is an excellent way to titrate the amount of central stimulant medication necessary to treat these patients, marked pupillary fatigue oscillations are seen in narcoleptic patients and in patients with ADHD. The oscillations will be reduced in a measurable way and help determine when the right dose of medication is reached.

OCULAR DISEASES—Almost every patient with an ocular disease will benefit from an examination utilizing the above system and method. One important test of the pupils in eye care is the “swinging flashlight test”. A PLR is elicited in one eye and after the recovery phase the flashlight is rapidly swung over to the other fellow eye and the initial pupillary diameter is assessed. If the pupil now dilates instead of contracting, with the same light illumination going into the other eye, it is diagnosed that there is a defect in the light transmission to the midbrain, somewhere in the pathway from the retina-to-the optic nerve-to-the optic tract-to-the midbrain. This defect in light perception by the midbrain, causing the pupil to not constrict as much on the diseased side, is in the afferent pathway to the brain. (It will not occur with an opacity in the media of the eye, which is what you see through as opposed to what actually perceives light and images, the retina, such as a cataract or blood in the vitreous jelly or with amblyopia, a lazy eye which is otherwise without disease).

It is called an Afferent Pupillary Defect (APD). Within the midbrain there are numerous cross-connections from the right to the left side of the neurons in the midbrain that initiate the constriction of the pupil in both eyes, so when light is shined in one eye, both eyes contract equally. This is called the consensual light reflex. This is why we need only one eye to see the PLR, and if the pupils are unequal, we should look at both independently. Diseases that affect the afferent pathway, retinal detachments, optic nerve diseases such as multiple sclerosis, glaucoma, that occur in only one eye or affect one eye more than the other, will have a positive APD.

If the initial movement of the second pupil is to dilate and not constrict, the APD is positive. If the initial diameter or area of the pupil in the second eye is greater than the diameter or area in the first eye, the APD is positive. This is an excellent clinical test but difficult to quantitate. Is there a positive APD? Sometimes it is equivocal. The system will make this a more quantifiable test.

The majority of the nerve fibers from the retina that travel back to the midbrain come from the macula, the center of the retina, where most of our useful vision is centered, man is predominantly a macular animal. An APD is known to be present with a substantial amount of retinal destruction such as in a retinal detachment. The system should be able to detect more subtle changes in the relative transmission of the macula to the midbrain with our APD test.

Alternatively, there may also be small movements of the eyes as macular degeneration increases. The most sensitive part of the macula, where you “fixate”, can change as the disease progresses. If a new area is now the most sensitive part we could detect a change in “fixation”, a shift in the alignment of the eyes. This would require the patient fixing on a word or object and determining the center of the pupil in both eyes (at a fixed distance) and seeing if it changes.

The system should be able to analyze macular degeneration in patients where we take an initial scan and determine the alignment of the eyes and follow this over time (stored in the cloud for each patient) to see if it changes. A change in fixation determined by a shift in the alignment of the eyes may signify progression of their macular degeneration and patients would be directed to see their eye doctor. There is a current test of patients looking at a grid to see if straight lines are distorted and if the distortion changes over time, the Amsler Grid.

TUMORS—Tumors act in two ways, different nerve paths or nerve centers can be invaded by the tumor directly or these paths (axons) or centers (neurons or collections of them known as ganglia) are damaged secondarily by pressure from the mass. By looking at more than the PLR alone, diplopia (misalignment of the eyes at a time which we can see by looking at both eyes with video), ptosis (drooping of one lid greater than the other), nystagmus, gaze palsies (palsies of upward gaze and convergence are common in midbrain tumors as is horizontal and rotary nystagmus) and other disorders of ocular motility as well as pupillary disturbances, including analyzing the light reaction in both eyes. Prior art devices look at only one eye, while the system disclosed herein captures both eyes so it will better diagnose tumors.

The diagnosis of Homer's Syndrome (ptosis, miosis and enophthalmos) will be facilitated with the system as we will have images of both eyes, allowing determination of lid position and possible proptosis (exophthalmos) (outward bulging of the eye) or enophthalmos (inward retraction of the globe of the eye).

TRAUMA—Post traumatic trauma can cause pupillary signs through varying and different mechanisms including damage to the afferent and efferent light reflex pathways, damage to the sympathetic pupillodilator outflow or to the pupillary centers in the brainstem and these can be in combination. This damage can be produced directly by mechanical force at the time of injury of secondarily by one or more pathologic mechanisms:

Hemorrhage;

Edema;

Consequent Brain Shifts and herniations;

Vascular Stenosis or Thrombosis;

Air or Fat Emboli; and

Ischemia.

PUPILARY SIGNS, POST-TRAUMATICALLY—may be useful by revealing organic defects in patients in whom the condition had been dismissed as psychoneurotic. Post-traumatic sequelae often develop after head trauma, even when the patient seems, at first, to be uninjured. If pupillary fibers in the 3^{rd} nerve are damaged with trauma, they can regenerate in a haphazard fashion and show signs of “misdirection”. The reflex pattern can look like an Argyll Robertson pupil (light reflexes lost but the pupil constricts briskly with near vision effort) and also when the globe is adducted or even in up and down movements which can also cause the upper lid to raise. Such pupils can be distorted, ectopic or both and the shape can change when the eye is moved with gaze because of the uneven pattern of regeneration. The system described herein, with its ability to track eye movements will be better able to identify this.

Trauma can affect the postganglionic pupillodilator neuron at several sites along its intracranial course by compression within the carotid canal where the fibers are spun around the artery and by skull fracture involving the temporal bene or the middle fossa and the ophthalmic division of the fifth nerve (Raeder's Trigeminal Syndrome) or the 5th and the 6th nerve (Gradenigo's syndrome) or even damage to the 3,4,5,6,7 or 8th nerves. Also damage to the cavernous sinus or the area of the superior orbital fissure can involve damage to the sympathetic fibers together with the third or nerves.

It was well known by the turn of the last century that mydriasis (dilation of the pupil) was an ominous sign. In most cases there was hemorrhage on the side of the large pupil and immediate surgery was needed to save the patient's life. Head trauma that resulted in bilaterally large, fixed (nonreactive to light) pupils was almost invariably fatal. When a patient was observed soon after an accident, it could be seen that a moderate constriction preceded enlargement of the ipsilateral pupil; and when the same sequence of events developed a little later in the second eye a severe hemorrhage was in progress. This involves the dynamics of pressure induced behavior of the brain and its vessels and the role of the limiting effects of volumes of the skull and the dural septa.

As a tumor or hemorrhage in one of the cerebral hemispheres or any supratentorial mass lesion, enlarges, the brain is pushed to the opposite side of the skull. The brainstem is pushed sideways and with increasing downward and lateral pressure and the entire hippocampal gyrus may be forced into the tentorial gap. The third nerve is then injured in several ways. In patients with supratentorial mass lesions the pupils can be important indicators of impending disaster. Unilateral mydriasis may precede all other physical signs, for example, in patients with slowly developing epi- or subdural hematomas. These can result from apparently trivial trauma, and the pupil sign may give the first warning of serious trouble brewing. In fact, it is encouraged that no mydriatics be used for fundal examinations when an obtunded patient is admitted for observation. There are diagnostic methods such as the EEGG, brain scan, arteriogram ST scan MRI and ultrasound that my help to make the diagnosis in these case, but there may be no time or facilities (on an ambulance) available for these tests and immediate evaluation of the pupils together with an examination of the respiratory, cardiovascular, metabolic and neurologic systems clinically may give clues as to whether the patient is getting worse or improving.

Sympathetic deficits can occur from trauma to the spinal cord, its ventral roots, the upper chest (including traumatic pneumothorax), the brachial plexus, or the sympathetic nerves in the neck and the involved pupil will show characteristic defects in the PLR of sympathetic paralysis, all in the dark-adapted state, during contractions to light the difference decreases, it increases during redilation and psychosensory reflex dilation is poor on the affected side. This can occur from any trauma to these areas including traffic accidents, blows to the head, diving accidents, trauma to the neck (whiplash type of extension -flexion injury) and even chiropractic manipulation.

Pupillary signs can distinguish between post traumatic syndromes, as an indicator of organic fatigue distinct from depression. Pupillary findings may indicate that the patient's complaints are related to organic brain damage rather than to be purely psychoneurotic in nature.

Patients with a post-concussion syndrome have been found to have a general hyperreflexia with large pupils and inhibited PLR, a sign confirming the organic nature of their nervousness and hyperexcitability. Other patients will have excessive pupillary “fatigue waves” indicating that their condition of fatigue or mental depression (also associated with pupillary signs unstable W or V shaped PLR).

We have no anatomical findings to explain either the increased central inhibition or the enhanced central fatigue in these patients. However, these pupillary signs are unconscious and involuntary; they certainly cannot be produced by the patient in the hope of successful mitigation. They are objective indicators—quite unknown to the patient—of exaggerated responsiveness (or of increased vulnerability) of neurons located in the diencephalic—mesencephalic border zone of the midbrain. These findings agree with the fact that short- or long-lasting brainstem findings are common after head trauma: coma, nausea, and vomiting; a slow, bounding or rapid pulse with alterations in blood pressure and respiration; disturbances of sleep and of temperature regulation; and changes in water, salt, fat and other metabolisms. An influence of the upper brainstem upon these mechanisms is well documented physiologically and pathologically.

Aneurysms of the aortic arch and carotid artery as well as intracranial aneurysms of the carotid artery and its branches or of the vertebro-basilar arterial tree can involve the pupilloconstrictor fibers of the 3rd nerve or the postganglionic pupillodilator fibers in the sympathetic system. The pupil can be used to distinguish among different types of migraine, especially between cluster headaches and the ophthalmologic migraine.

PSYCHIATRIC CONDITIONS—Schizophrenia have long been known to cause decreased pupillary unrest (increased oscillations) with pupillary dilation, suppression of the PLR and reduced reflex dilation. These oscillations can be related to physical and emotional stress, increased emotional tension or excitement of the patients compared to normal rather than to the loss of cerebral impulses. The reactions are inappropriate and excessive for the patient's age the system disclosed herein will be helpful to analyze these responses.

Patients with catatonic schizophrenia have a reaction pattern of the pupils that includes:

Large dilated pupils;

Pupillary Unrest is absent (No Oscillations);

The pupils react poorly to psychosensory stimulation, no reflex pupillary dilation;

Temporary and complete abolition of the light reflex;

Also seen in hysterical patients who throw themselves into fits and in epileptic seizures; and

This is not observed in patients with depressive psychoses.

Loewenfeld (Loewenfeld, Irene E., The Pupil, Butterworth Heineman, Boston, Mass., 1999 at 777) found manic depressive patients also hypersensitive to psychosensory stimulation; and some showed spontaneously increased central inhibition of the light reflex. But this was less common than in schizophrenia; and marked suppression of the light reflex is not part of the typical picture for manic-depressive illness. In fact, reduced central inhibition is commonly found in these patients (small pupils, vigorous reflex pupillary dilation with improvement of the PLR). In Loewenfeld's experience, similar pupillary signs may occur during both depressed and manic phases of the disease. Accordingly, she feels that the pupils do not indicate shifts in autonomic balance related to positive or negative mood changes in manic-depressive illness as has been claimed.

While the ultimate cause of these pupillary phenomena in the mentally ill is unknown, the physiologic mechanism is not difficult to demonstrate. It is the same that is normally evoked by any form of arousal, namely, simultaneous activation of sympathetic and inhibition of parasympathetic outflow to the iris. Except during acute excitement the inhibitory mechanism is the more important in schizophrenia.

This was shown when infrared-sensitive recording devices became available: in darkness the pupils of schizophrenic patients actually are no larger than those of age-matched control persons, but their reactions to light are less extensive. The mydriasis described by so many authors in the past was only apparent: when examined in light these pupils seemed enlarged because they constricted less extensively to illumination than did the pupils of normal individuals.

Results of pharmacologic work fits well with these findings. Treatment with anti-psychotic drugs like trifluorperazine or haloperidol contracts the patient's pupils only slightly (in darkness), but they bring about distinct enhancement of the PLR. Conversely, small dosed of amphetamines in normal subjects have only minimal mydriatic effect (in darkness) but they cause strong inhibition of the PLR and they reduce the extent and frequency of pupillary oscillations in steady light.

Pupillary reactions during shock treatments is the same as during convulsive seizures. It can also be used as a guide during stereotactic neurosurgery.

DRUG REACTIONS—Pupils have been used to monitor the effects of systemic drugs. This includes antipsychotic drugs as well as stimulants and psychosis-inducing drugs like LSD or mescaline. Their potency of different drugs can be established objectively and their mechanism, as far as the autonomic nervous system is concerned, can be revealed. Sedation or excitement as detrimental side effects of drug treatment for psychiatric as well as systemic diseases, such as treating hypertension, can also be evaluated by recording pupillary behavior.

The Pupil in Cognitive Studies—There are findings of objective differences on the PLR associated with mental processing of information between normal people and patients with psychiatric illness. When presented choices in conditions determined to be “certain” versus “uncertain”, in normal subjects the pupils will dilate in uncertain situations. Schizophrenic patient have only minimal dilation in these situations are presented as stimuli. Patients with mental depression do not exhibit this lack of differentiation between certain (or highly probable) and uncertain (or rare) stimuli, although their reactions were not as extensive as those of normal control subjects.

Fatigue—Characteristic pupillary fatigue waves are seen in normal people after prolonged, exhausting stress, and patients whose fatigue has been ascribed to neurotic tendencies but have had for example crushing head injuries or narcolepsy exhibit the same “fatigue waves” as normal fatigued individuals.

Neuroses—The neurotic patient's personality and general reactions are commonly mirrored in the pupillary reflex pattern. Moderate loss of central inhibition was almost universal in these patients and “see-saw” anisocoria (a difference in the size between the 2 pupils of greater than 0.3 mm, that can be transient, can change from one pupil to another, last for a variable period of time, and is likely caused by a shifting, asymmetric, central inhibition of the Edinger-Westphal nucleus) is present in more than ½ the patients. These patients frequently have physical signs referable to the autonomic nervous system, sleep disturbances, cardiovascular irregularities, abnormal sweating, and gastrointestinal and other dysfunctions.

Autonomic Attacks—See hypoglycemic reaction.

THE PUPILS IN COMA AND DEATH—Cheyne-Stokes respiration is an alternating pattern of apnea and hyperapnea found in many patients with a terminal illness, especially lung, kidney, central nervous system. Periodic breathing can also be brought about by metabolic dysfunctions such as uremia and by central nervous system depressing drugs such as opioids.

1. The pupils enlarge during the respiratory phases of the cycle, dilation may precede the first breath by some seconds, and the pupils contract when the breathing wanes;

2. The pupils become extremely small during the apneic phases, the PLR has often been said to have been completely lost. However, because of the tight miosis, residual PLR may have been overlooked;

3. The efferent mechanism of the pupillary dilations during the respiratory phases is simultaneous excitation of the dilator muscle accompanied by inhibition of pupilloconstrictor neurons in the midbrain, while the pupillary contractions during the apneic phases are due to the decline of these sympathetic ad central inhibitory impulses; and 4. The pupillary oscillations are part of intermittent arousal reactions triggered by the medullary reticular formation in response to anoxia, on a background of physiologically or pathologically reduced consciousness. But in pathologic cases the respiratory, pupillary, cardiovascular, somatic, and mental components of the reaction may be fragmented, depending on the cause and the level of unconsciousness, and in neurogenic cases upon the extent and location of the lesion.

In deep coma the pupils are generally small, just as they are in deep narcosis. They can be enlarged by strong sensory stimulation; but unless the patient (or animal) is actually awakened, no sympathetic activity is elicited, and the pupillary dilation is due to inhibition of the sphincter nucleus alone.

At the moment of death the pupillary sphincter relaxes and (at least if death is sudden) there is a wave of sympathetic discharges to the dilator. The wide mydriasis which results is followed, during the next 2-48 hours, by slow re-contraction, leaving the pupils somewhat smaller than they had been during life when the individual was awake. There are wide variations in the amplitude and timing of these events between individuals and depending on the conditions at the moment of death.

COMA—It has been known since ancient times that in coma, the pupils are small. They resemble in all respects the pupils of normal individuals under the influence of deep sleep or anesthesia or of patients with brainstem damage such as pontine lesions. Comatose or deeply anesthetized individuals can be aroused to a degree by strong sensory stimulation; and this is accompanied by slow, incomplete enlargement of the pupils. But unless the subject actually awakens, sympathetic outflow to the pupillary dilator muscle is not evoked by sensory stimulation. The mechanism for the slow enlargement of the pupils is central inhibition of the sphincter. While it has been said that the PLR is abolished in comatose patients, the tight miosis makes further constriction minimal and slow and easily overlooked. Furthermore, if vigorous sensory stimulation is applied, the pupils will enlarge and the PLR will become apparent.

BRAIN DEATH—Because of advanced life support systems, the cessation of breathing and circulation and death of the brain can be drawn out virtually indefinitely. As for the pupils, there problems with fixed and dilated pupils as a certain sign of brain death in certain circumstances and the system and method may correlate better with SPO2 levels and with brain death.

SURGICAL PROCEDURES—The system can be used during surgical procedures to diagnose inadvertent cutting or trauma to pupillary neurons for example during thyroid surgery or to the ciliary ganglion by retrobulbar injections. Impairment of ocular blood flow by radical neck dissections or dental procedures. Anoxia during anesthesia, air emboli, neurosurgical procedures such a lumbar punctures or myelograms, birth trauma (Klumpke's Syndrome of brachial plexus injury).

LAW ENFORCEMENT—The system includes an ability to analyze eye movements including gaze-shifting movements such as smooth pursuit and saccades. It enables the system to detect alcohol and will aid in the detection of other drugs.

### EXAMPLE

FIG. 8 is a graph depicting an actual PLR (Loewenfeld, Irene E, (1999), The Pupil, Boston, Mass., Butterworth Heineman, page 777) showing the concentration of Benzedrine (brand name under which amphetamine was marketed in the U.S. by Smith, Kline & French, now part of GlaxoSmithKline, London, UK) administered, which at 10 mg is low and has almost no sympathomimetic effect but does have a marked central effect on the pupils. The PLR after administration of the drug (broken lines) is shifted upwards and the reactions are enhanced as the increased pupil size creates a larger mechanical range for contraction. Also, the oscillations, or fatigue waves, are abolished with the amphetamine induced central stimulation. If a similar sympathomimetic drug topically were administered to the eye, with no central effect on the brain, the pupil diameter would enlarge, but there would be no effect on the fatigue waves or pupillary oscillations.

One or more embodiments of the present invention have been described. Nevertheless, it will be understood that various modifications may be made without departing from the spirit and scope of the invention. Accordingly, other embodiments are within the scope of the following claims.

What is claimed is:

1. A system to record and evaluate a mammalian eyeball response to a stimulus and diagnose a medical condition therefrom, the system comprising:
a handheld device that includes:
a video recorder effective to captures a plurality of images of one or both eyeballs;
a first non-transient digital memory and a handheld processor configured to provide real-time guidance to maximize resolution of the video recorder;
a communication port effective to transmit the plurality of images to a remote server and to receive data from the remote server; and
the remote server having:
a remote communication port effective to receive the plurality of images and to transmit data to the handheld device; and
a second non-transient digital memory and a remote processor configured to extract data from the plurality of images and process that data to diagnose the medical condition.

2. The system of claim 1 wherein the plurality of images are temporally sequential.

3. The system of claim 2 wherein the video recorder captures images at a rate of from 10 to 100 frames per second.

4. The system of claim 3 wherein the video recorder captures images at a rate of from 15 to 60 frames per second.

5. The system of claim 3 wherein the first non-transient digital memory and the handheld processor are configured to extract one or more features from the plurality of images.

6. The system of claim 5 wherein a feature extracted from the plurality of images is a boundary between a pupil and an iris of the mammalian eyeball.

7. The system of claim 6 wherein the boundary is determined by distinguishing the pupil from the iris.

8. The system of claim 7 wherein the pupil is distinguished from the iris based on color density.

9. The system of claim 7 wherein the pupil is distinguished from the iris based on neural network image processing.

10. The system of claim 7 wherein the first non-transient digital memory and the handheld processor are configured to measure a pupillary feature selected from the group consisting of pupil diameter, ratio of pupil diameter to iris diameter, ratio of pupil area to iris area, pupil area, eyeball movement and combinations thereof.

11. The system of claim 7 wherein metadata associated with a particular frame includes temporal location of the frame and the value of the pupillary feature extracted from the image stored within that frame.

12. The system of claim 6 wherein the real-time guidance positions the handheld device to minimize shadows and reflections overlying the boundary.

13. The system of claim 12 wherein the real-time guidance non-invasively spaces the handheld device from 2 to 8 inches from the eyeball.

14. The system of claim 13 wherein the real-time guidance non-invasively spaces the handheld device a nominal 3 inches from the eyeball.

15. The system of claim 1 wherein the stimulus is a flash of visible light.

16. The system of claim 15 wherein the flash of visible light has a duration of from 0.1 second to 1.5 seconds.

17. The system of claim 5 wherein the feature extraction is applied to fewer than all images.

18. The system of claim 17 wherein the feature extraction is applied to each nth frame where “n” is an integer greater than 1.

19. The system of claim 18 wherein “n” is 4.

20. The system of claim 10 wherein the remote server is configured to process the pupillary feature to identify a neurological condition.

21. The system of claim 20 wherein the neurological condition is identified on the brain-side of the blood/brain barrier.

22. The system of claim 21 wherein the neurological condition is due to intake of a chemical substance or due to a disease.

23. The system of claim 22 wherein the pupillary feature is input into a neural network having nodes corresponding to pupillary response to a chemical substance or a disease.

24. The system of claim 23 wherein each node corresponds to a pupillary response based on a concentration and identity of at least one chemical substance.

25. The system of claim 24 wherein the output of the neural network identifies one or more chemical substances.

26. The system of claim 25 wherein the remote server is configured to transmit the identity of the one or more chemical substances to the handheld device.

27. A handheld device configured to record a mammalian eyeball response to a stimulus comprising:
a video recorder effective to capture a plurality of images of one or both eyeballs;
a non-transient digital memory and a processor configured to provide real-time guidance to maximize resolution of the video recorder; and
a communication port effective to transmit the plurality of images to a remote server and to receive data from the remote server.

28. The handheld device of claim 27 wherein the plurality of images are temporally sequential.

29. The handheld device of claim 28 wherein the video recorder captures images at a rate of from 10 to 100 frames per second.

30. The handheld device of claim 29 wherein the video recorder captures images at a rate of from 15 to 60 frames per second.

31. The handheld device of claim 29 wherein the non-transient digital memory and the processor are configured to extract one or more features from the plurality of images.

32. The handheld device of claim 31 wherein a feature extracted from the plurality of images is a boundary between a pupil and an iris of the mammalian eyeball.

33. The handheld device of claim 32 wherein the boundary is determined by distinguishing the pupil from the iris.

34. The handheld device of claim 33 wherein the pupil is distinguished from the iris based on color density.

35. The handheld device of claim 33 wherein the non-transient digital memory and the processor are configured to measure a pupillary feature selected from the group consisting of pupil diameter, ratio of pupil diameter to iris diameter, ratio of pupil area to iris area, pupil area, eyeball movement and combinations thereof.

36. The handheld device of claim 33 wherein metadata associated with a particular frame includes temporal location of the frame and the value of the pupillary feature extracted from the image stored within that frame.

37. The handheld device of claim 32 wherein the real-time guidance directs that the handheld device be positioned to minimize shadows and reflections overlying the boundary.

38. The handheld device of claim 37 wherein the real-time guidance directs that the handheld device be non-invasively spaced from 2 to 8 inches from the eyeball.

39. The handheld device of claim 27 wherein the stimulus is a flash of visible light.

40. The handheld device of claim 39 wherein the flash has a duration of from 0.1 second to 5 seconds.

41. The handheld device of claim 31 wherein the feature extraction is applied to less than all images.

42. The handheld device of claim 41 wherein the feature extraction is applied to each nth frame where “n” is an integer greater than 1.

43. The handheld device of claim 42 wherein “n” is 4.

44. A method for treating a mammalian subject suffering from a chemical substance overdose, the method comprising the steps of:
stimulating one or both eyeballs of the mammalian subject;
capturing a response to that stimulus as a plurality of images and extracting image data with a handheld device;
transmitting the plurality of images and image data associated with the images to a remote server;
determining one or more pupillary light reflex measurements from the plurality of images and from the image data;
processing the pupillary light reflex measurements to identify the one or more chemical substances; in the subject, wherein the extracting and processing are performed on the remote server;
transmitting the identity of the one or more chemical substances to the handheld device; and
administering a treatment consistent with the presence of the one or more chemical substances identified.

45. The method of claim 44 including temporally sequencing the plurality of images.

46. The method of claim 45 including capturing images at a rate of from 10 to 100 frames per second.

47. The method of claim 46 including the step of extracting one or more features from the plurality of images.

48. The method of claim 47 including selecting a feature extracted from the plurality of images to be a boundary between a pupil and an iris of the mammalian eyeball.

49. The method of claim 48 wherein the boundary is determined by distinguishing the pupil from the iris.

50. The method of claim 49 wherein the pupil is distinguished from the iris based on color density.

51. The method of claim 49 wherein the pupil is distinguished from the iris based on neural network image processing.

52. The method of claim 49 including measuring a pupillary feature selected from the group consisting of pupil diameter, ratio of pupil diameter to iris diameter, ratio of pupil area to iris area, pupil area, eyeball movement and combinations thereof.

53. The method of claim 49 wherein metadata associated with a particular frame includes temporal location of the frame and the value of the pupillary feature extracted from the image stored within that frame.

54. The method of claim 48 wherein real-time guidance directs that the handheld device be positioned to minimize shadows and reflections overlying the boundary.

55. The method of claim 44 wherein the stimulus is a flash of visible light.

56. The method of claim 55 wherein the flash has a duration of from 0.1 second to 1.5 seconds.

57. The method of claim 47 wherein the feature extraction is applied to fewer than all images.

58. The method of claim 57 wherein the feature extraction is applied to each nth frame where “n” is an integer greater than 1.

59. The method of claim 52 wherein the remote server is configured to process the pupillary feature to identify one or more chemical substances.

60. The method of claim 59 including inputting the pupillary feature into a neural network having nodes corresponding to pupillary response to a chemical substance.

61. The method of claim 60 wherein each node corresponds to a pupillary response based on a concentration and identity of at least one chemical substance.

62. The method of claim 61 wherein the output of the neural network identifies one or more chemical substances.
